(19) United States
(12) Patent Application Publication
(10) Pub. No.: US 2015/0257759 A1
(43) Pub. Date: Sep. 17, 2015

(54) IMPLANT FOR FACILITATING SUTURED SIDE-TO-SIDE ARTERIOVENOUS FISTULA CREATION AND MAINTAINING PATENCY

(71) Applicant: Cook Medical Technologies LLC, Bloomington, IN (US)

(72) Inventors: Adam Shields, Lafayette, IN (US); Susie Vogel, Lafayette, IN (US)

(21) Appl. No.: 14/658,851

(22) Filed: Mar. 16, 2015

Related U.S. Application Data
(60) Provisional application No. 61/952,984, filed on Mar. 14, 2014.

(51) Int. Cl. A61B 17/11; A61M 1/36

(52) U.S. Cl. CPC A61B 17/11; A61M 1/3655; A61B 2017/1107; A61B 2017/1139

(57) ABSTRACT

An anastomosis device for facilitating side-to-side arteriovenous fistula creation and maintenance of patency thereof is presented. The device comprises two device halves each containing an anastomosis window which is in fluid connection with an interior lumen of the device. Further, the device comprises a pair of suturing windows in which the bounds of the fistula are created. The invention also has an embodiment wherein a method of joining a vein and an artery in order to create a fistula is presented.

### CROSS REFERENCE TO RELATED APPLICATIONS

This application claims the benefit of U.S. Provisional Patent Application No. 61/952,984, filed on Mar. 14, 2014, the entire contents of which is hereby incorporated by reference.

### BACKGROUND OF THE INVENTION

1. Field of Invention

The present invention relates to medical devices. More particularly, the invention relates to an implant which in one embodiment facilitates sutured side-to-side arteriovenous fistula (AVF) creation and maintains the patency thereof.

2. Background

End-stage renal disease (ESRD) is a growing problem in the United States and abroad, with the number of patients requiring treatment far outstripping the number of donor kidneys available. Those patients who are unable to receive a kidney transplant are treated by dialysis, with roughly ten times as many patients receiving hemodialysis as all other forms combined.

To minimize treatment time, hemodialysis requires a large blood volume flow rate. Increasing flow is typically achieved through the surgical creation of an arteriovenous shunt. This creates a low resistance pathway, significantly increasing flow through a graft or an arteriovenous fistula.

In practice, AVF is preferred to graft usage because fistulas have better long-term patency rates and reduced incidences of secondary interventions after creation. However, the surgical creation of an AVF and the subsequent venous tissue remodelling required to realize optimized flow rates is only successful in approximately half of surgical procedures. Failures involving AVF are largely due to stenosis via neointimal hyperplasia and thrombosis. A potential cause of neointimal hyperplasia is the exposure of venous tissue to the abnormal hemodynamic conditions resulting from significantly increased flow rates and pulsatility of the added arterial blood flow. In other cases, large flow rates can cause extreme dilation and result in oversized fistulas which fail to achieve the purpose for which they were created.

There exists a need for an improved method of creating arteriovenous fistula and maintaining the patency thereof, including implantation of devices designed to achieve these purposes.

### BRIEF SUMMARY OF THE INVENTION

The present invention provides a pair of device halves, each device half having a proximal end and a distal end, each of the device halves having an anastomosis window formed therethrough defining a first arch and a second arch opposite the first arch, the first and second arches being disposed normal to the proximal and distal ends, each device half having an inner surface and an outer surface opposite the inner surface, the device halves being connected at a proximal connector portion and at a distal connector portion on the inner surfaces defining a space therebetween and such that the anastomosis windows are in alignment with each other, the proximal connector portion and the distal connector portion defining a first suturing window and a second suturing window opposite the first suturing window, the first and second suturing windows being normal the anastomosis windows.

In another embodiment, the present invention provides a pair of device halves, each device half having a proximal end and a distal end, each of the device halves having an anastomosis window formed therethrough, the anastomosis window defining a first arch and a second arch opposite the first arch, the first and second arches being disposed normal to the proximal and distal end, each device half having an inner surface and an outer surface opposite the inner surface, the device halves being connected at a proximal connector portion and at a distal connector portion on the inner surfaces defining a space therebetween such that the anastomosis windows are in alignment with each other, the proximal connector portion and the distal connector portion defining a first suturing window and a second suturing window opposite the first suturing window, the first and second suturing windows being normal the anastomosis windows, each device half having a first flange proximally extending from the proximal end, each device half having a second flange distally extending from the distal end, the first flanges being spaced apart from each other defining a first flange gap, the second flanges being spaced apart from each other defining a second flange gap, each device half comprising a plurality of barbs attached to the first and second arches on the inner surface, each device half comprising a plurality of suture ports formed through the first flanges and the second flanges.

In another embodiment, the present invention provides a method of facilitating side-to-side fistula along a longitudinal portion of a vein and an artery, the vein having a vein wall, the artery having an artery wall. The method comprises in one step incising the vein wall substantially longitudinally to define a vein aperture, the vein wall comprising a first vein lip and a second vein lip opposite the vein aperture from the first vein lip. In a second step the method comprises incising the artery wall substantially longitudinally to define an artery aperture, the artery wall comprising a first artery lip and a second artery lip opposite the artery aperture from the first artery lip. The method includes implanting an anastomosis device in accordance with the principles of the present invention to form the side-to-side fistula. The implantation procedure includes disposing the second artery lip over one second arch and the second vein lip over the opposite second arch such that the second arches are disposed within the vein wall and the artery wall and such that the second vein lip and the second artery lip are disposed in the second suturing window; joining the second vein lip to the second artery lip by a first surgical method; disposing the first artery lip over one first arch and the first vein lip over the opposite first arch such that the first arches are disposed within the vein wall and the artery wall and such that the first vein lip and the first artery lip are disposed in the first suturing window; and joining the first vein lip to the first artery lip by a second surgical method.

Further objects, features, and advantages of the present invention will become apparent from consideration of the following description and the appended claims when taken in connection with the accompanying drawings.

### BRIEF DESCRIPTION OF THE DRAWINGS

FIG. 1A is diagram of major arteries and veins of the human arm and selected arteriovenous fistulas that may be created therein;

FIG. 1B is a diagram of blood flow through an arteriovenous fistula surgically created in a human forearm;

FIG. 2A-2C are schematic representations of end-to-end, side-to-end, and side-to-side arteriovenous fistulas, respectively;

FIG. 3 is a perspective view of one embodiment of a fistula stabilization device;

FIG. 4 is a top view of the fistula stabilization device of FIG. 3;

FIG. 5A-5B are schematic representations of two embodiments of anastomosis windows of fistula stabilization devices;

FIG. 6 is another embodiment of a fistula stabilization device with additional structural features incorporated; and

FIG. 7A-E are schematic views of the steps of one embodiment of a fistula stabilization device insertion procedure.

### DETAILED DESCRIPTION OF THE INVENTION

The following provides a detailed description of currently preferred embodiments of the present invention. The description is not intended to limit the invention in any manner, but rather serves to enable those skilled in the art to make and use the invention.

In this description, when referring to a device, the term distal is used to refer to an end of a component which in use is furthest from the physician during the medical procedure, including within a patient. The term proximal is used to refer to an end of a component closest to the physician and in practice in or adjacent an external manipulation part of the deployment or treatment apparatus. Similarly, when referring to an implant such as an anastomosis device the term distal is used to refer to an end of the device which in use is furthest from the physician during the medical procedure and the term proximal is used to refer to an end of the device which is closest to the physician during the medical procedure.

The terms “substantially” or “about” used herein with reference to a quantity includes variations in the recited quantity that are equivalent to the quantity recited, such as an amount that is equivalent to the quantity recited for an intended purpose or function. “Substantially” or derivatives thereof will be understood to mean significantly or in large part.

In this disclosure, where directionality is addressed, a “top” element may also be a “first” element without being a top relative to another component, and a “bottom” element may also be a “second” element in likewise fashion.

The present disclosure generally provides an implant or device which facilitates the formation of an arteriovenous fistula and maintaining the patency thereof. Surgical fistula creation is presently the preferred method of increasing blood flow for end stage renal disease (ESRD) patients who are receiving hemodialysis. The advantages of fistulas over such treatments as grafts are numerous. For instance, of the treatment methods for ESRD patients that might be used to increase blood flow rates, arteriovenous fistulas (AVFs) are associated with decreased morbidity and mortality, and have the superior primary patency rates, the lowest rates of thrombosis, and require the fewest secondary interventions. AVFs generally provide longer hemodialysis access survival rates. Medical data shows that the total number of interventions during the life of the access is considerably lower for AVFs compared with AV grafts and that AVFs have lower rates of infection than AV grafts. The danger of infection is also decreased upon successful formation of an AVF. Thus, it is not surprising that AVFs also lead to lower hospitalization rates among ESRD patients who undergo some form of treatment to increase blood flow rates in order to facilitate hemodialysis.

However, there is a need to improve the ways that AVFs are created and maintained. Fewer than 15% of dialysis fistulas remain patent and can function without problems during the entire period of a patient's dependence on hemodialysis. The mean problem-free patency period after creation of native fistulas is approximately 3 years, whereas prosthetic polytetrafluoroethylene (PTFE) grafts last 1-2 years before indications of failure or thrombosis are noted. After multiple interventions to treat underlying stenosis and thrombosis, the long-term secondary patency rates for native fistulas are reportedly 7 years for fistulas in the forearm and 3-5 years for fistulas in the upper arm. Prosthetic grafts remain patent for up to 2 years.

For prosthetic grafts, fistula failure and eventual occlusion occur most commonly as a result of the progressive narrowing of the venous anastomosis; for native fistulas, failure occurs most commonly as a result of the narrowing of the outflow vein. The primary underlying pathophysiologic mechanism responsible for causing the failure is intimal hyperplasia at the anastomotic site. Additional causes include surgical and iatrogenic trauma, such as repeated venipunctures. Stenoses along the venous outflow and in intragraft locations (for prosthetic PTFE grafts) are also common.

The embodiments of the device described herein are designed in part to overcome these deficiencies. The anastomosis device provides a luminal region with a defined geometry for blood flow therethrough. It also provides a limited amount of contact between the intimal surfaces of the vessels to be connected by the fistula relative to methods of directly connecting the artery and the vein surgically.

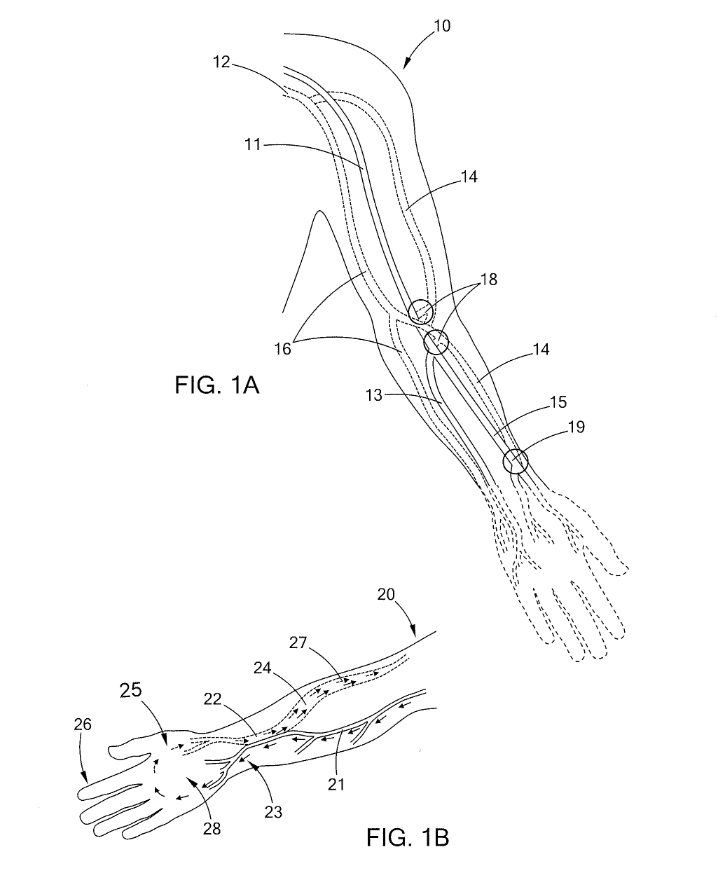

Referring to FIG. 1A, the veins and arteries of the arm are illustrated. Fistulas in ESRD patients are generally created in the arm. Arm 10 contains a plurality of arteries (illustrated in solid lines) and veins (illustrated in dashed lines.) Brachial artery 11 originates toward the shoulder and splits into an ulnar artery 13 and a radial artery 15 in the region of the elbow. Likewise, axillary vein 12 and cephalic vein 14 run through the shoulder region, and in the area of the elbow, the axillary vein 12 splits into the basilic vein 16. The vessels that were previously split, arteries and veins alike, can undergo anastomosis and form fistulas. Exemplary fistulas illustrated include brachial-cephalic arteriovenous fistula 18, between the brachial artery 11 and the cephalic vein 14, and radial-cephalic fistula 19, between the radial artery and the cephalic vein 14.

FIG. 1B illustrates an example of the blood flow consequences of arteriovenous fistula creation. Artery 21 carries arterial blood 23 away from the heart and through the arm 20 in the direction of hand 26. Arteriovenous fistula 29 connects the artery 21 to vein 22. A portion of the blood flows through fistula 29 and the remainder continues on its natural path through the capillary system 28 in the hand. In the capillaries the blood deoxygenates and flows as venous flow 25 through the vein 22. Due to the fistula, a combined arterial-venous blood flow 27 forms, and as a result of the increased volume and flow rate of blood through the vasculature, an enlarged portion 24 of the vein arises.

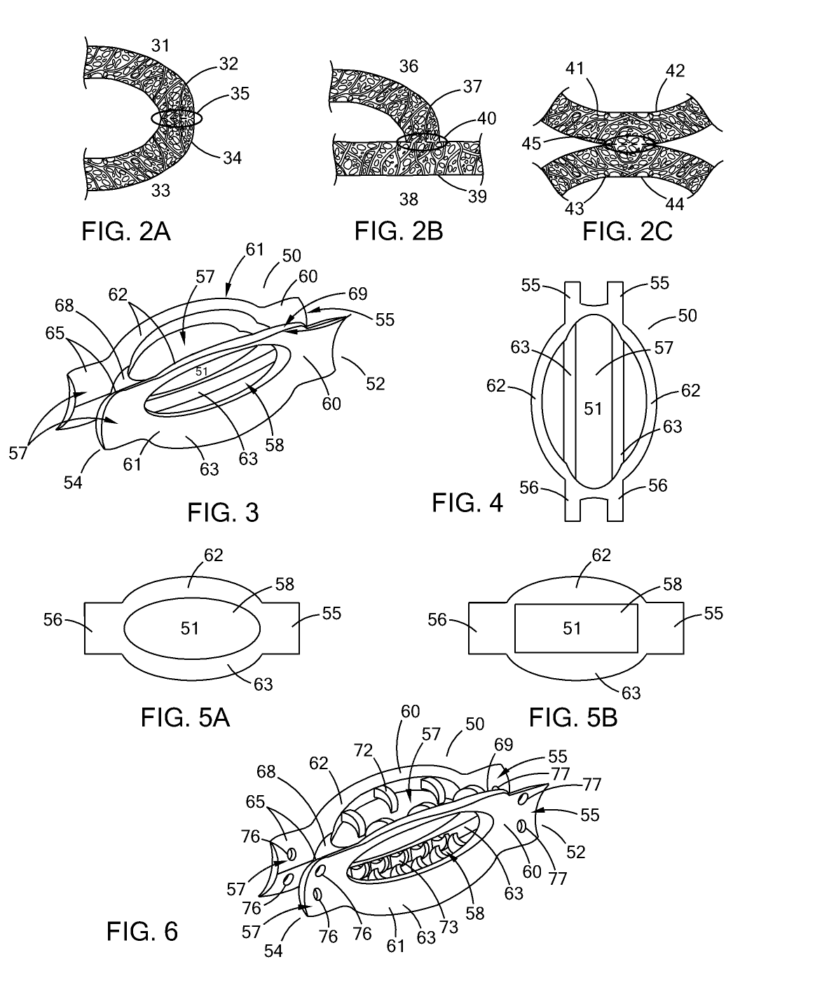

FIGS. 2A-C are examples of different configurations of arteriovenous fistula. FIG. 2A illustrates an artery 31 with an artery end 32 as well as a vein 33 having a vein end 34. The artery end 32 is joined to vein end 34 to form end-to-end arteriovenous fistula 35. Such a fistula configuration can be technically difficult to execute and has an intrinsic disadvantage as artificial creation of an end-to-end fistula naturally requires that the vessels used in its formation be completely severed in order to connect the ends, thereby completely disconnecting the vessels from the rest of the vascular system.

FIG. 2B instead shows artery 38 with artery end 39 and vein 36 with vein wall 37. The artery end 39 is attached to vein 36 through vein wall 38 to form side-to-end arteriovenous fistula 40. This type of fistula is technically simpler to execute for a surgeon but still has the drawback that the vessel whose end it used (in the case illustrated, artery 38 with artery end 39) has its downstream regions disconnected from the rest of the vascular system. This complete rerouting of the blood flow can have negative consequences.

An alternative fistula arrangement, the side-to-side arteriovenous fistula, is illustrated in FIG. 2C. In this example, artery 41 has been perforated through artery wall 42 and joined to vein 43 through vein wall 44 to create side-to-side fistula 45. Because of the side-to-side configuration, a portion of the blood continues to flow in the natural pattern to downstream tissues, unlike the case in which an artery end or a vein end is employed to create the anastomosis. However, even creation of this type of AVF can be difficult using standard surgical techniques. A properly-designed device can simplify the procedure and, importantly, maintain patency of the AVF.

FIG. 3 illustrates a device 50 in accordance with one embodiment of the present invention. Device 50 has a proximal end 52 and a distal end 54. It should be noted, however, that because of the symmetry between the proximal ends and distal ends, that these designations are arbitrary and employed for purposes of convenience.

The device is made of two device halves 60. In the embodiment illustrated in FIG. 3, the device halves 60 extend between proximal end 52 and distal end 54, as well as first arches 62 and second arches 63, which serve to connect the proximal ends 52 to the distal ends 54. The first arches 62 and the second arches 63 also frame an empty space in each device half 60 which is anastomosis window 58. The device halves 60 are opposite and in alignment with one another, and the first arch 62 of one of the device halves are opposite and in alignment with the first arch 62 of the opposite device half, and the second arch 63 is opposite and in alignment with the second arch 63 of the other device half. Between the device halves 60 and in fluid connection with anastomosis windows 58 is a central space of the device which is device lumen 51.

The device halves 60 have an inner surface which provides a boundary for the device lumen 51 and also have a portion that does not face the device lumen 51. This latter portion is referred to as outer device surface 61. Outer device surface 61 is encompassed by portions of first arch 62 and second arch 63 and all other portions of device half 60 that are opposite to the device lumen 51. Outer device surface 61 curves away from the device lumen 51, giving each device a “C” shape when viewed head-on and giving the device 50 a butterfly shape when viewed in profile.

The embodiment of device 50 shown in FIG. 3 further comprises a pair of flanges at each end. A device in accordance with the present disclosure does not require a flange. Each device half 60 has a first flange 55 at proximal end 52 and a second flange 56 at distal end 54. The first flange 55 and the second flange 56 extend in the proximal and distal directions, respectively, beyond the first arch 62, the second arch 63, and the anastomosis window 58. The first flange 55 of one device half 60 is not in contact with the first flange 55 of the other device half 60, being separated by inner flange gap 65. The second flanges 56 are constructed in a similar manner, also being separated and having an inner flange gap 65 separating the two second flanges 65.

As mentioned previously, when there are no features of the device which impart a directionality or introduce an asymmetrical element to it, and the device is outside a surgical context (that is, it is not being used at that moment in a procedure or has not been implanted into a patient), the terms proximal and distal can be switched with one another. The terms top and bottom can be switched in a similar fashion. However, the outer portion of the device, including outer device surfaces 61, will remain an outer or exterior portion, and the device lumen 51 will remain interior, regardless of the designation of top/bottom and proximal/distal.

An anastomosis device 50 with the structural features described herein can be made of a number of different materials. Such a device 50 can be made of a biocompatible and biologically-inert material which will be well-tolerated by the tissues it contacts but will not encourage, for instance, growth of new intimal tissue across its openings. The device 50 can be made of a variety of polymers, including photosensitive polymers which are used for rapid prototyping applications. The polymers can have a stiffness ranging from relatively labile to relatively stiff, taking into account that the integrity of the anastomosis windows 58 must be maintained for optimal operation of the device. The device 50 may also be coated or impregnated with drugs which will prevent or slow endothelialization of the anastomosis windows 58 and thereby reduce the space available for blood flow through the AVF. The device may be made by a number of processes, including injection molding.

The slits to be cut into the artery walls and vein walls to provide openings for blood flow, when used in a device in accordance with an embodiment of the present invention will be made parallel to the direction of blood flow or substantially longitudinally. Put another way, the slitting is done parallel to a portion of a longitudinal axis of the vessel to be slit. Thus a longitudinal portion of a vein or of an artery comprises a portion which is parallel to the direction of blood flow, and can be in a plane substantially normal to the longitudinal axis. In many cases such a longitudinal portion may be about one centimeter in length. Therefore, a device 50 will have an anastomosis window 58 which extends for slightly greater than one centimeter in the proximal-to-distal dimension. It is possible that a favorable increase in blood flow through an AVF can be achieved with a fistula which is less than one centimeter wide, in which case it will be acceptable to construct a device 50 with an anastomosis window 58 which is less than 1 centimeter wide as well. Contrarily, certain patients may require that an AVF longer than one centimeter wide be constructed. In such cases, a device which has a longer anastomosis window 58 than one centimeter will be best suited for facilitating hemodialysis in such patients. The overall diameter of the device, as measured from one anastomosis window to another, may be about five to ten millimeters across.

In one embodiment the anastomosis windows are opposite and in alignment with one another across the body of the device such that blood may freely flow through the device. The first and second arches can be disposed normal to the proximal and distal ends of the device such that the planes of the proximal end or the distal end are substantially perpendicular to a plane that can be drawn through at least a portion of the arches. The first suturing window may be opposite the second suturing window and these may be disposed substantially normal the anastomosis windows. Thus there may be four planar faces of the device situated approximately 90 degrees from one another, each containing an anastomosis or a suturing window.

Referring now to FIG. 4, a top view of the device 50 is provided. From this perspective, the first flanges 55 can be seen in connection with the first arches 62, which further connect to the second flanges 56. The first flanges 55 are also connected to the second flanges by second arches 63. The distal portions of the first arches 62 and the second arches 63 meet at distal connecting portion 69 and the proximal ends of these meet and join at proximal connecting portion 68. These connecting portions sit opposite of the outer device surfaces 61. They function to connect the device halves 60. The proximal connecting portion 68 provides a bound at the proximal end of the device 50 for the interior device lumen 51 and the distal connecting portion 69 provides a bound at the distal end for the device lumen 51. This further defines the flow area of blood through the AVF and prevents flow into the intervascular space. The proximal connector portion 68 and the distal connector portion 69 may take on similar shapes; in some cases, they can be wedge-shaped connectors which taper as they approach the ends of the device. In other cases they may be three-dimensional solids of varying shapes. In still other embodiments the device halves may simply connect at an edge or a line, which edge or line would comprise the connector portion. Finally, the connector portions may simply comprise a point at which the device halves meet.

Between the first arches 62 and bounded by the proximal connecting portion 68 and the distal connecting portion 69 is a space which is the first suturing window 57. The second arches 63, the proximal connecting portion 68, and the distal connecting portion 69 frame a second suturing window. The first suturing window 57 and the second suturing window provide a top and bottom access, respectively, to the device lumen 51. The suturing windows are the spaces in which the vessels being manipulated to create the AVF will be sutured or otherwise connected together to form the top and bottom portions of the fistula.

FIG. 5A-5B are side views of a device half 60 in accordance with embodiments of the present invention. These views illustrate how the first arch 62 and the second arch 63 define the bounds of the anastomosis window 58. The anastomosis window 58 can take on a variety of shapes. In the preferred embodiment of FIG. 5A, the anastomosis window 58 has a substantially elliptical shape. The dimensions of the ellipse confer certain advantages on the device. For instance, the larger axis of the ellipse provides a way of incorporating the entire length of the AVF which is formed from combining an aperture in the vein and an aperture in the artery after slitting the vessels within the anastomosis window 58, while the smaller axis of the ellipse further defines the geometry of the anastomosis and ensures that the dimensions of the device do not greatly exceed the height of the vessels involved. Likewise, a device according to the embodiment of FIG. 5B, in which the anastomosis window 58 is a rectangle having a greater length than height dimension, would confer similar advantages to the elliptical window of FIG. 5A. These embodiments, however, are not intended to be limiting. Devices having anastomosis windows 58 in various shapes, including squares, circles, or other polygonal shapes are also envisioned as acceptable alternative embodiments to those illustrated.

In another embodiment of the device 50 in accordance with the principles of the present invention, the device of FIG. 6 is shown. Notably, the device 50 depicted herein has a plurality of top barbs 72 which assist in the gripping and stabilization of portions of vessel walls which have been pulled through the device lumen 51 and into the first suturing window 57. The top barbs 72 are formed along a portion of first arch 62 and extend with their points within device lumen 51. A barb can have two ends, including the end which comprises a portion of the device from which it extends, and the barb can then extend to a second end, which may be a point. Each point is capable of securing the vascular material but are not sharp enough to pierce and damage it. The barbs may curve and have a dentite or bladelike shape. A barb that curves inward is considered to extend in a roughly opposite direction from the outer surface of the device half from which it originates and toward the opposite device half. Such a barb may be integrally formed with the device halves, such as by an additive manufacturing process, including 3D printing, and formed unitarily with the remainder of the device. A device with top or first barbs 72 could possibly ease and accelerate the suturing process.

Another embodiment has a plurality of bottom or second barbs 73 which assist in the gripping and stabilization of portions of vessel walls which have been pulled through the device lumen 51 and into the second suturing window. Analogously to top or first barbs 72, bottom or second barbs 73 are formed along a portion of second arch 63 and extend with their points within device lumen 51. The points are capable of securing the vascular material but are not sharp enough to pierce and damage it. A device may possess only top barbs 72, only bottom barbs 73, both top barbs 72 and bottom barbs 73, or no barbs at all.

The embodiment of device 50 illustrated in FIG. 6 also possesses a plurality of distal suture ports 76 at the distal end 54 and a plurality of proximal suture ports at proximal end 52. In the embodiment illustrated, the distal suture ports 76 are formed within the second flanges 56, through outer device surface 61 and connecting to inner flange gap 65. However, in embodiments which lack flanges, these suture ports may be formed at the respective device end at any point wide enough to accommodate them. The suture ports 76 and 77 are configured to permit suturing of the device 50 to the vascular tissue to ease handling and provide stability through the implantation process.

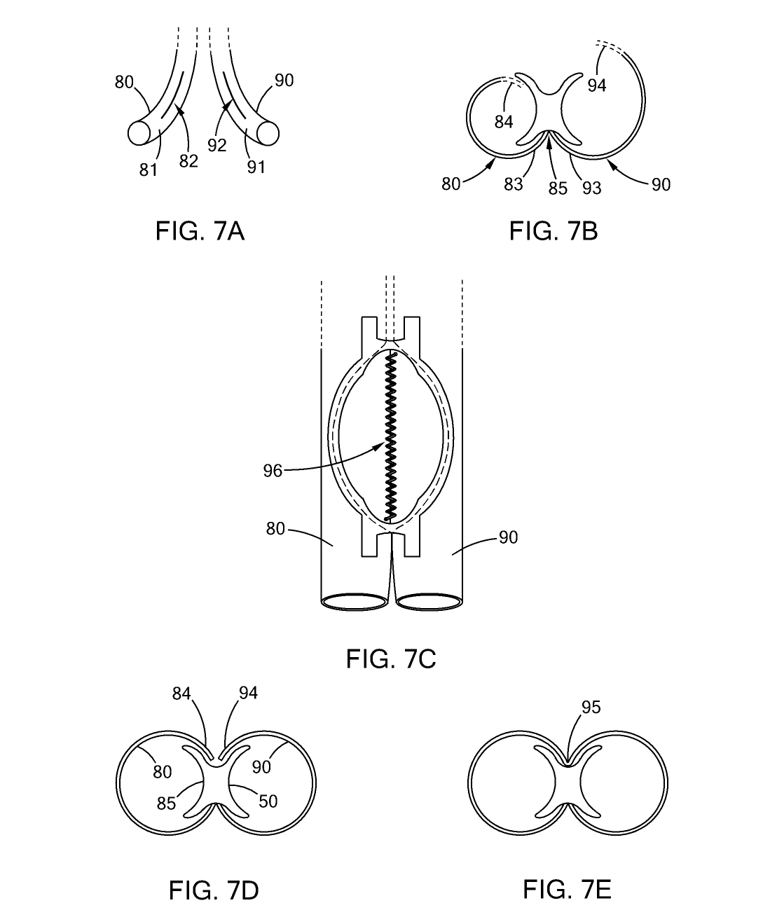

Referring now to FIG. 7A-7E, a method of creating an arteriovenous fistula in accordance with one embodiment of this invention is illustrated. A person having skill in the art will appreciate that variations to the process are possible beyond what is illustrated in FIG. 7A-7E without deviating from the spirit of the present invention.

FIG. 7A illustrates a vein 80 and an artery 90 which are to be used in the creation of an AVF. The vein 80 is bounded by vein wall 81 and the artery 90 by artery wall 91. A vein slit 82 forming a vein aperture has been formed through vein wall 81 and an artery slit 92 creating an artery aperture has been formed through artery wall 91. The slits may be made by any acceptable means, including by scalpel.

Referring now to FIG. 7B, the vessels have been opened. The vein 80 has been opened along vein slit 82 to divide the vein 80 into second vein lip 83 and top or first vein lip 84 such that the second vein lip 83 lies opposite the first vein lip 84. Likewise, the artery 90 has been opened along artery slit or aperture 92 to create top or first artery portion 94 and bottom artery lip 93. The bottom vein portion 83 and the bottom or second artery lip 93 have been inserted into the bottom suturing window of device 50. The position at which the vessels meet is first arteriovenous junction 85.

Referring to FIG. 7C, a top view of the procedure in progress is illustrated. Looking through the first suturing window 57, the bottom vein lip 83 and the bottom artery portion 93 have been joined at first arteriovenous junction 85 by a first surgical step, in this case by suture 96. The suturing can be done through the first suturing window 57 and may be stitched or otherwise attached in any suitable manner, for instance using a surgical glue such as fibrin glue.

FIG. 7D illustrates a fourth step in the AVF creation procedure. In this step the top vein lip 84 and the top artery portion 94 have been inserted through the first suturing window 57 and into device lumen 51 of device 50. If the embodiment of the device has a plurality of top barbs 72 as illustrated in FIG. 6, the top vein portion 84 and the top artery lip 94 are secured thereon.

FIG. 7E illustrates the final step in placement of the device 50. The top vein portion 84 and the top artery lip 94 meet at second arteriovenous junction 95. The second arteriovenous junction 95 can be secured in the same way as the first arteriovenous junction 85 or it can be sealed in a different manner. The AVF has been created and blood is flowing through the anastomosis window from the artery 90 into the device lumen 51 and then into the vein 80.

As mentioned, further steps may be incorporated into the method in accordance with other embodiments of the present invention. For instance, in the case of a device having at least one suture port, the vein 80 or artery 90 or both can be secured to the device 50 by suturing through the vessel walls and the ports. The vessels will then be thus secured to the device 50 and spaced apart from one another. Additionally, if the device 50 contains a plurality of top barbs 72 or a plurality of bottom barbs 73, the respective vessel portions may be secured thereon at the appropriate steps of the procedure.

As a person skilled in the art will readily appreciate, the above description is meant as an illustration of the implementation of the principles of this invention. This description is not intended to limit the scope or application of this invention in that the invention is susceptible to modification variation and change, without departing from the spirit of this invention, as defined in the following claims.

1. A device for facilitating side-to-side arteriovenous fistula comprising:
a pair of device halves, each device half having a proximal end and a distal end, each of the device halves having an anastomosis window formed therethrough defining a first arch and a second arch opposite the first arch, the first and second arches being disposed normal to the proximal and distal ends, each device half having an inner surface and an outer surface opposite the inner surface, the device halves being connected at a proximal connector portion and at a distal connector portion on the inner surfaces defining a space therebetween and such that the anastomosis windows are in alignment with each other, the proximal connector portion and the distal connector portion defining a first suturing window and a second suturing window opposite the first suturing window, the first and second suturing windows being normal the anastomosis windows.

2. The device of claim 1 wherein the first suturing window is bounded by the proximal connector portion, the distal connector portion, and the inner surfaces of the second arches of each device half.

3. The device of claim 1 wherein the second suturing window is bounded by the proximal connector portion, the distal connector portion, and the inner surfaces of the first arches of each device half.

4. The device of claim 1 wherein the proximal ends of each device half define a first flange extending beyond the proximal connector portion, and wherein the distal end of each device half defines a pair of distal flanges extending beyond the distal connector portion.

5. The device of claim 4 wherein the first flanges are spaced apart and define a first flange gap therebetween, and wherein the second flanges are spaced apart and define a second flange gap therebetween.

6. The device of claim 1 further comprising a plurality of barbs attached to the inner surface of each first arch, the barbs having points biased toward the device half opposite that from which the barbs extend.

7. The device of claim 1 further comprising a plurality of barbs attached to the inner surface of each second arch, the barbs having points biased toward the device half opposite that from which the barbs extend.

8. The device of claim 1 further comprising a plurality of suture ports at the proximal and distal ends.

9. The device of claim 5 further comprising a plurality of suture ports through the proximal and second flanges.

10. The device of claim 1 comprising substantially elliptical anastomosis windows.

11. The device of claim 1 comprising substantially rectangular anastomosis windows.

12. A device for facilitating side-to-side arteriovenous fistula comprising:
a pair of device halves, each device half having a proximal end and a distal end, each of the device halves having an anastomosis window formed therethrough, the anastomosis window defining a first arch and a second arch opposite the first arch, the first and second arches being disposed normal to the proximal and distal end, each device half having an inner surface and an outer surface opposite the inner surface, the device halves being connected at a proximal connector portion and at a distal connector portion on the inner surfaces defining a space therebetween and such that the anastomosis windows are in alignment with each other, the proximal connector portion and the distal connector portion defining a first suturing window and a second suturing window opposite the first suturing window, the first and second suturing windows being normal the anastomosis windows, each device half having a first flange proximally extending from the proximal end, and a second flange distally extending from the distal end, the first flanges in alignment with and being spaced apart from each other defining a first flange gap, the second flanges in alignment with and being spaced apart from each other defining a second flange gap, each device half comprising a plurality of barbs attached to the first and second arches on the inner surfaces, each device half comprising a plurality of suture ports formed through the first flanges and the second flanges.

13. A method of facilitating side-to-side fistula along longitudinal portions of a vein having a vein wall and an artery having an artery wall, the method comprising:
incising the vein wall substantially longitudinally to provide a first vein lip and a second vein lip, defining a vein aperture;
incising the vein wall substantially longitudinally to provide a first artery lip and a second artery lip, defining an artery aperture;
implanting an anastomosis device to form the side-to-side fistula, the anastomosis device comprising a pair of device halves, each device half having a proximal end and a distal end, each of the device halves having an anastomosis window formed therethrough, defining a first arch and a second arch opposite the first arch, the first and second arches being disposed normal to the proximal and distal ends, each device half having an inner surface and an outer surface opposite the inner surface, the device halves being connected at a proximal connector portion and at a distal connector portion on the inner surfaces defining a space therebetween and such that the anastomosis windows are in alignment with each other, the proximal connector portion and the distal connector portion defining a first suturing window and a second suturing window opposite the first suturing window, the first and second suturing windows being normal the anastomosis windows, each of the first arches being disposed opposite each other, each of the second arches being disposed opposite each other to facilitate side-to-side fistula.

14. The method of claim 13 wherein implanting comprises:
disposing the second artery lip over one of the second arches and the second vein lip over the opposite second arch, the second vein lip and the second artery lip being disposed in the second suturing window such that one of the second arches is disposed within the artery wall and the opposite second arch is disposed within the vein wall;
joining the second vein lip to the second artery lip;
disposing the first artery lip over one of the first arches and the first vein lip over the opposite first arch, the first vein lip and the first artery lip being disposed in the first suturing window such that one of the first arches is disposed within the artery wall and the opposite first arch is disposed within the vein wall; and
joining the first vein lip to the first artery lip.

15. The method of claim 13 further comprising providing a device having a plurality of barbs attached to the inner surface of each second arch of the device and each first arch of the device.

16. The method of claim 14 comprising the additional step of placing the bottom vein lip and the bottom artery lip to a portion of the device interior to the plurality of barbs attached to the second arch of the device and securing the bottom artery lip and the bottom vein lip within the device.

17. The method of claim 15 further comprising the additional step of placing the bottom vein lip and the bottom artery lip to a portion of the device interior to the plurality of barbs attached to the second arch of the device and securing the bottom artery lip and the bottom vein lip within the device.

18. The method of claim 11 further comprising providing a device having suture ports substantially at the proximal end and distal end of the device, the artery and the vein being secured to the device by suturing through the suture ports.

19. The method of claim 11 wherein joining the vein lips to the artery lips comprises suturing.

20. The method of claim 11 wherein joining the vein lips to the artery lips comprises applying surgical glue.
